SEQUENCE LISTING (1 sequence)

SEQ ID NO: 1 - protein, 306 aa, SARS coronavirus
SGFRKMAFPSGKVEGCMVQVTCGTTTLNGLWLDDTVYCPRHVICTAEDMLNPNYEDLLIRKSNHSFLVQAGNVQLRVIGHSMQNCLLRLKVDTSNPKTPKYKFVRIQPGQTFSVLACYNGSPSGVYQCAMRPNHTIKGSFLNGSCGSVGFNIDYDCVSFCYMHHMELPTGVHAGTDLEGKFYGPFVDRQTAQAAGTDTTITLNVLAWLYAAVINGDRWFLNRFTTTLNDFNLVAMKYNYEPLTQDHVDILGPLSAQTGIAVLDMCAALKELLQNGMNGRTILGSTILEDEFTPFDVVRQCSGVTFQ

(19) United States
(12) Patent Application Publication
(10) Pub. No.: US 2006/0014821 A1
(43) Pub. Date: Jan. 19, 2006

(54) Inhibitors of SARS 3C like protease

(75) Inventors: Mingying He, San Diego, CA (US); Robert Steven Kania, San Diego, CA (US); Jihong Lou, San Diego, CA (US); Simon Planken, San Diego, CA (US)

Correspondence Address: AGOURON PHARMACEUTICALS, INC., 10777 SCIENCE CENTER DRIVE, SAN DIEGO, CA, 92121, US

(73) Assignee: Agouron Pharmaceuticals, Inc.

(21) Appl. No.: 10/918,612

(22) Filed: Aug. 13, 2004

Related U.S. Application Data
(60) Provisional application No. 60/498,472, filed on Aug. 27, 2003.

(51) Int. Cl. A61K 31/4015; C07D 207/26

(52) U.S. Cl. 514/424; 548/550

(57) ABSTRACT

The invention relates to methods of inhibiting SARS-related coronavirus viral replication activity comprising contacting a SARS-related coronavirus protease with a therapeutically effective amount of a SARS 3C like protease inhibitor, and compositions comprising the same.

This application claims the benefit of U.S. Provisional Application No. 60/498,472, filed Aug. 27, 2004, the disclosures of which are incorporated herein by reference in their entireties.

### BACKGROUND OF THE INVENTION

The invention relates to compounds and methods of inhibiting Severe Acute Respiratory Syndrome viral replication activity comprising contacting a SARS-related coronavirus 3C-like proteinase with a therapeutically effective amount of a SARS 3C-like protease inhibitor. The invention further relates to pharmaceutical compositions containing the SARS 3C like proteinase inhibitor in a mammal by administering effective amounts of such coronavirus 3C like proteinase inhibitor.

A worldwide outbreak of Severe Acute Respiratory Syndrome-related coronavirus (“SARS”) has been associated with exposures originating from a single ill health care worker from Guangdong Province, China. Recently, the causative agent has been identified as a novel coronavirus. There is an acute need in the art for an effective treatment for the SARS-related coronavirus.

Recent evidence strongly implicates a new coronavirus as the causative agent of SARS (CDC). Coronavirus replication and transcription function is encoded by the so-called “replicase” gene (Thiel, Herold et al. 2001), which consists of two overlapping polyproteins that are extensively processed by viral proteases. The C-proximal region is processed at eleven conserved interdomain junctions by the coronavirus main or “3C-like” protease (Ziebuhr, Snijder et al. 2000). The name “3C-like” protease derives from certain similarities between the coronavirus enzyme and the well-known picornavirus 3C proteases (Gorbalenya, Koonin et al. 1989). These include substrate preferences, use of cysteine as an active site nucleophile in catalysis, and similarities in their putative overall polypeptide folds. Very recently Hilgenfeld and colleagues published a high-resolution X-ray structure of the porcine transmissible gastroenteritis coronavirus main protease (Anand, Palm et al. 2002). Atomic coordinates are available through the Protein Data Bank under accession code 1LVO.

For almost 10 years, researchers have been engaged in an effort to discover and develop drugs with utility for treating the common cold by targeting a key enzyme in rhinovirus replication, namely the 3C protease (Matthews, Smith et al. 1994). The picornaviruses are a family of tiny non-enveloped positive-stranded RNA-containing viruses that infect humans and other animals. These viruses include the human rhinoviruses, human polioviruses, human coxsackieviruses, human echoviruses, human and bovine enteroviruses, encephalomyocarditis viruses, meningitis virus, foot and mouth viruses, hepatitis A virus, and others. Picornaviral infections may be treated by inhibiting the proteolytic 3C enzymes. These enzymes are required for the natural maturation of the picornaviruses. They are responsible for the autocatalytic cleavage of the genomic, large polyprotein into the essential viral proteins. Members of the 3C protease family are cysteine proteases, where the sulfhydryl group most often cleaves the glutamine-glycine amide bond. Inhibition of 3C proteases is believed to block proteolytic cleavage of the polyprotein, which in turn can retard the maturation and replication of the viruses by interfering with viral particle production.

### SUMMARY OF THE INVENTION

The present invention relates to compounds of the formula 1 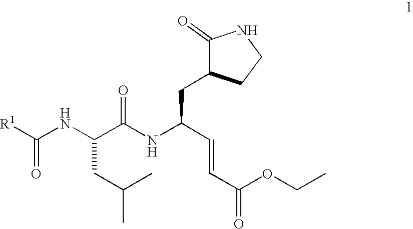
and to pharmaceutically acceptable salts and solvates thereof, wherein:
R¹ is selected from C₁ to C₄ alkyl; 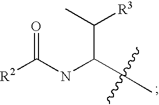
R² is selected from C₁ to C₄ alkyl and —OR⁵;
   R⁵ is selected from C₁ to C₆ alkyl and
R³ is selected from halogen, —OH, —OR⁴ and C₁, to C₄ alkyl.

The present invention also relates to compounds of the formula 2 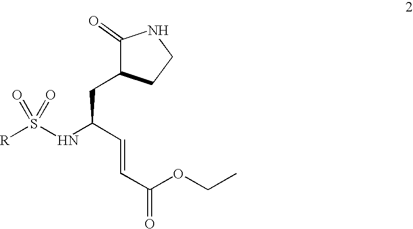
and to pharmaceutically acceptable salts and solvates thereof, wherein R is C₁ to C₁₀ alkyl, aryl, heteroaryl, C₅ to C₁₀ cycloalkyl and C₄ to C₉ heterocycloalkyl.

The present invention provides methods of inhibiting the activity of a coronavirus 3C protease (also known as proteinase), comprising contacting the coronavirus 3C protease with an effective amount of a SARS 3C protease inhibitor compound or agent.

The present invention provides a novel method of interfering with or preventing SARS viral replication activity comprising contacting a SARS protease with a therapeutically effective amount of a rhinovirus protease inhibitor.

In one embodiment of the present invention, the SARS coronavirus 3C-like protease inhibitor is administered orally or intravenously.

The present invention also provides a method of treating a condition that is mediated by coronavirus 3C-like protease activity in a patient by administering to said patient a pharmaceutically effective amount of a SARS protease inhibitor.

The present invention also provides a method of targeting SARS inhibition as a means of treating indications caused by SARS-related viral infections.

The present invention also provides a method of targeting viral or cellular targets identified by using rhinovirus inhibitors against SARS coronavirus 3C-like protease for treating indications caused by SARS-related viral infections.

The present invention also provides a method of identifying cellular or viral pathways interfering with the functioning of the members of which could be used for treating indications caused by SARS infections by administering a SARS protease inhibitor.

The present invention also provides a method of using SARS protease inhibitors as tools for understanding mechanism of action of other SARS inhibitors.

The present invention also provides a method of using SARS 3C like protease inhibitors for carrying out gene profiling experiments for monitoring the up or down regulation of genes for the purposed of identifying inhibitors for treating indications caused by SARS infections.

The present invention further provides a pharmaceutical composition for the treatment of SARS in a mammal containing an amount of a SARS 3C like protease inhibitor that is effective in treating SARS and a pharmaceutically acceptable carrier.

According to certain preferred embodiments of the above-described inventions, the SARS 3C like protease inhibitor is 4-(2-Acetylamino-4-methyl-pentanoylamino)-5-(2-oxo-pyrrolidin-3-yl)-pent-2-enoic acid ethyl ester.

According to certain preferred embodiments of the above-described inventions, the SARS 3C like protease inhibitor is 4-[2-(2-Acetylamino-3-hydroxy-butyrylamino)-4-methyl-pentanoylamino]-5-(2-oxo-pyrrolidin-3-yl)-pent-2-enoic acid ethyl ester.

According to certain preferred embodiments of the above-described inventions, the SARS 3C like protease inhibitor is 4-[2-(2-tert-Butoxycarbonylamino-3-methyl-butyrylamino)-4-methyl-pentanoylamino]-5-(2-oxo-pyrrolidin-3-yl)-pent-2-enoic acid ethyl ester.

According to certain preferred embodiments of the above-described inventions, the SARS 3C like protease inhibitor is 4-[2-(2-Acetylamino-3-methyl-butyrylamino)-4-methyl-pentanoylamino]-5-(2-oxo-pyrrolidin-3-yl)-pent-2-enoic acid ethyl ester.

According to certain preferred embodiments of the above-described inventions, the SARS 3C like protease inhibitor is 4-[2-(2-tert-Butoxycarbonylamino-3-hydroxy-butyrylamino)-4-methyl-pentanoylamino]-5-(2-oxo-pyrrolidin-3-yl)-pent-2-enoic acid ethyl ester.

According to certain preferred embodiments of the above-described inventions, the SARS 3C like protease inhibitor is 4-[2-Acetylamino-4-methyl-pentanoylamino)-4-methyl-pentanoylamino]-5-(2-oxo-pyrrolidin-3-yl)-pent-2-enoic acid ethyl ester.

### BRIEF DESCRIPTION OF THE FIGURES

FIG. 1 is a sequence alignment of 3C-like protein translated from SARS genome (AY274119) with TGEV 3C-like proteinase (1LVO) used for homology modeling. The location of the first indel was adjusted from the BLAST alignment to better reflect the multiple alignment of other coronavirus 3C-like proteins (Anand, Palm et al. 2002). 43% of the residues are identical in this alignment.

FIG. 2 depicts the twelve residues used to superimpose the 3C-like protein structures identified by visual inspection. They include a region near the catalytic cysteine, the catalytic histidine, and a region of structurally conserved beta-strand.

FIG. 3 is a homology model for SARS 3C-like protease (atom-color wire) superimposed on the cocrystal structure of rhinovirus 3C protease (purple wire) bound to AG7088 (atom-color stick).

FIG. 4 shows the hydrogen bond between AG7088 and rhinovirus 3C protease from the cocrystal structure (1CQQ), the corresponding hydrogen bonds between AG7088 and the model of SARS 3C protease when superimposed on the structure of rhinovirus 3C protease. Four of the hydrogen bonds predicted between AG7088 and the SARS 3C protease model are also found in the cocrystal structure of TGEV (1LVO), where water or the small molecule 2-methyl-2,4-pentanediol replace the inhibitor.

FIG. 5 shows solvent accessible (Connolly) surface of the binding site of AG7088 in the crystal structure of rhinovirus 3C protease (upper panel) and the corresponding surface in the SARS 3C protease model (lower panel).

FIG. 6 shows the percent (%) identity between coronavirus 3C proteases including SARS (AY274119), MHV: murine hepatitis virus (M55148), BCoV: bovine coronavirus (Q8V440), PEDV: porcine epidemic diarrhea virus (Q91AV2), FIPV: feline infectious peritonitis virus (Q98VG9), TGEV: transmissible gastroenteritis virus (Q9lW05), HCoV: human coronavirus 229E (Q9DLN0), AIBV: avian infectious bronchitis virus (M95169).

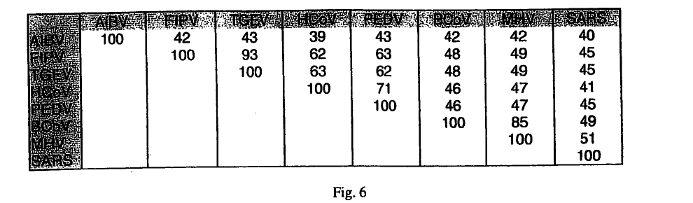

FIG. 7 is a phylogenetic tree describing the coronavirus 3C proteases.

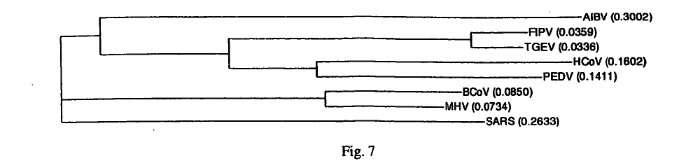

FIG. 8 is a molecular model of 4-[2-(2-tert-Butoxycarbonylamino-3-hydroxy-butyrylamino)-4-methyl-pentanoylamino]-5-(2-oxo-pyrrolidin-3-yl)-pent-2-enoic acid ethyl ester in the binding site of SARS 3C like protease.

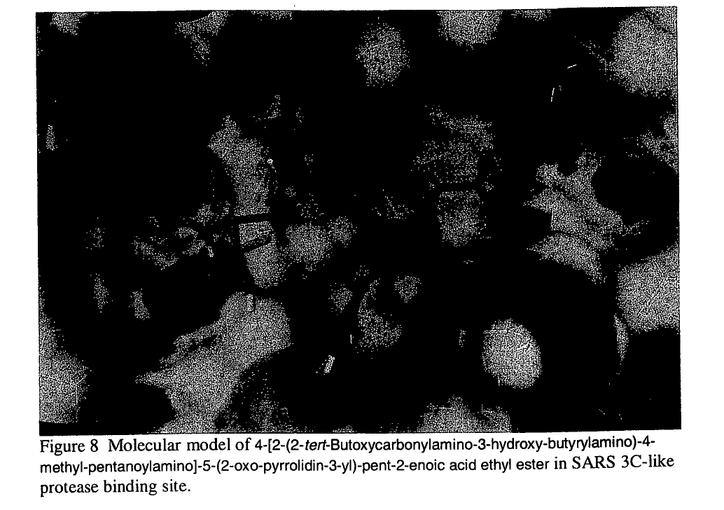

FIG. 9 is a molecular model of 4-[2-(2-tert-Butoxycarbonylamino-3-methyl-butyrylamino)-4-methyl-pentanoylamino]-5-(2-oxo-pyrrolidin-3-yl)-pent-2-enoic acid ethyl ester in the binding site of SARS 3C like protease.

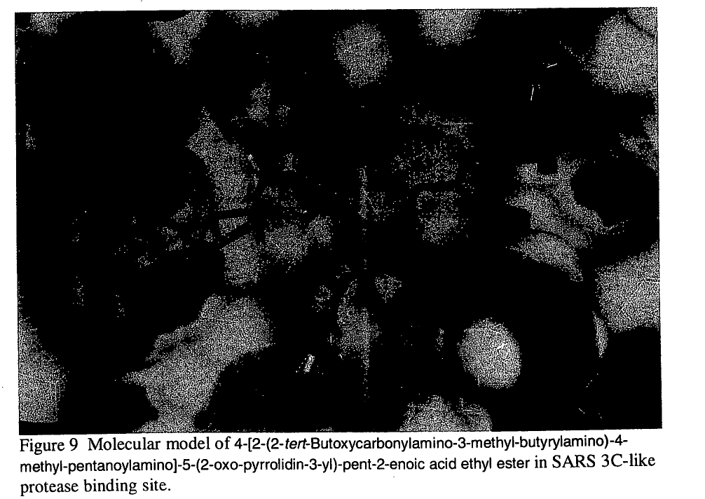

FIG. 10 is a molecular model of 4-(2-Acetylamino-4-methyl-pentanoylamino)-5-(2-oxo-pyrrolidin-3-yl)-pent-2-enoic acid ethyl ester in the binding site of SARS 3C like protease.

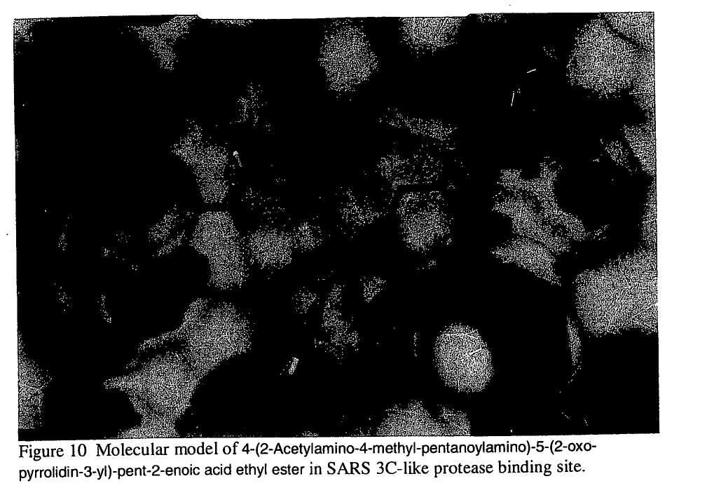

FIG. 11 is a molecular model of 4-[2-(2-Acetylamino-3-hydroxy-butyrylamino)-4-methyl-pentanoylamino]-5-(2-oxo-pyrrolidin-3-yl)-pent-2-enoic acid ethyl ester in the binding site of SARS 3C like protease.

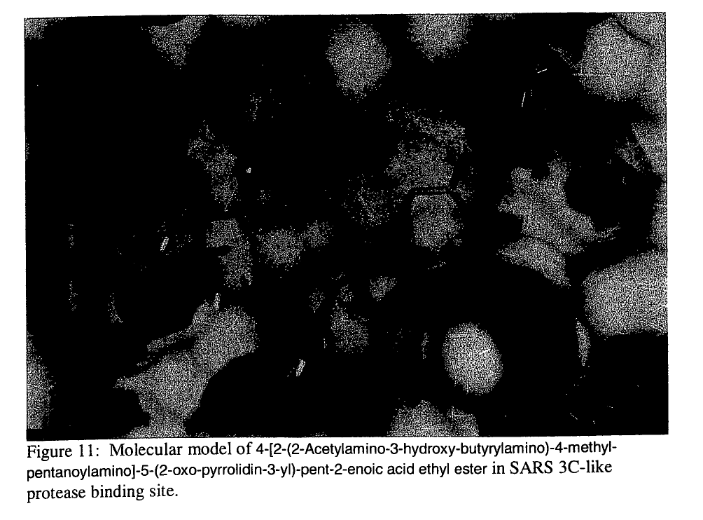

FIG. 12 is a molecular model of 4-[2-(2-Acetylamino-3-methyl-butyrylamino)-4-methyl-pentanoylamino]-5-(2-oxo-pyrrolidin-3-yl)-pent-2-enoic acid ethyl ester in the binding site of SARS 3C like protease.

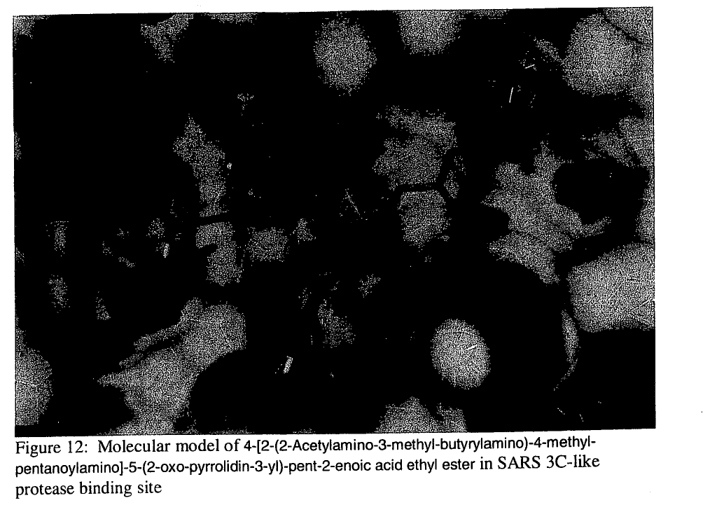

FIG. 13 is a molecular model of 4-[2-Acetylamino-4-methyl-pentanoylamino)-4-methyl-pentanoylamino]-5-(2-oxo-pyrrolidin-3-yl)-pent-2-enoic acid ethyl ester in the binding site of SARS 3C like protease.

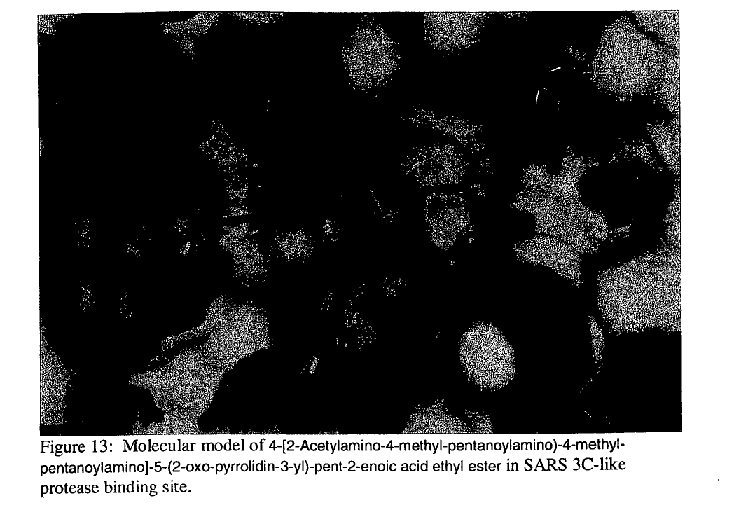

FIG. 14 is a molecular model of ethyl(2E,4S)-4-[(methylsulfonyl)amino]-5-[(3S)-2-oxopyrrolidin-3-yl]pent-2-enoate in the binding site of SARS 3C like protease.

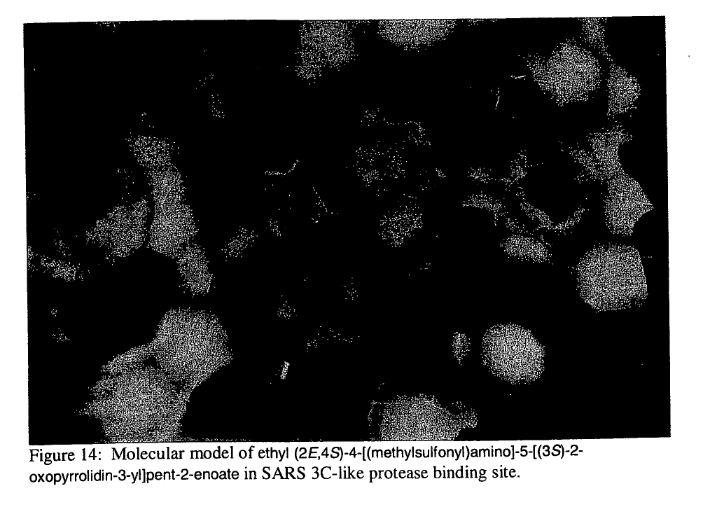

FIG. 15 is a molecular model of ethyl(2E,4S)-4-[(2-naphthylsulfonyl)amino]-5-[(3S)-2-oxopyrrolidin-3-yl]pent-2-enoate in the binding site of SARS 3C like protease.

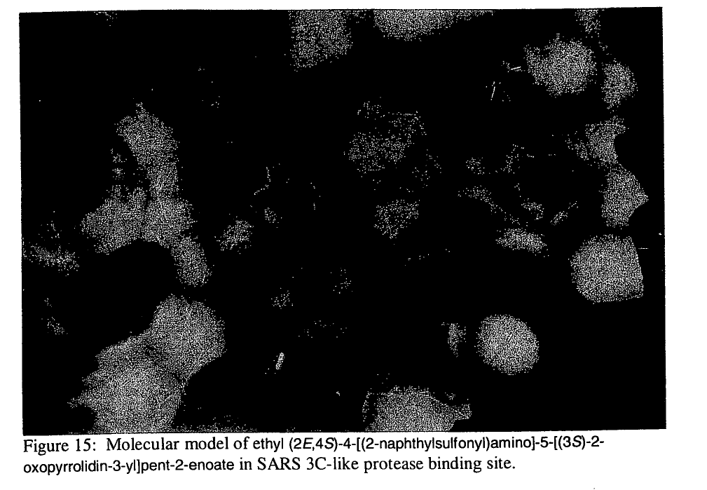

### DETAILED DESCRIPTION OF THE INVENTION AND PREFERRED EMBODIMENTS

For purposes of the present invention, as described and claimed herein, the following terms are defined as follows:

As used herein, the terms “comprising” and “including” are used in their open, non-limiting sense.

The term “halo”, as used herein, unless otherwise indicated, means fluoro, chloro, bromo or iodo. Preferred halo groups are fluoro, chloro and bromo.

The term “alkyl”, as used herein, unless otherwise indicated, includes saturated monovalent hydrocarbon radicals having straight or branched moieties.

The term “alkenyl”, as used herein, unless otherwise indicated, includes alkyl moieties having at least one carbon-carbon double bond wherein alkyl is as defined above and including E and Z isomers of said alkenyl moiety.

The term “alkynyl”, as used herein, unless otherwise indicated, includes alkyl moieties having at least one carbon-carbon triple bond wherein alkyl is as defined above.

The term “alkoxy”, as used herein, unless otherwise indicated, includes O-alkyl groups wherein alkyl is as defined above.

The term “Me” means methyl, “Et” means ethyl, and “Ac” means acetyl.

The term “cycloalkyl”, as used herein, unless otherwise indicated refers to a non-aromatic, saturated or partially saturated, monocyclic or fused, spiro or unfused bicyclic or tricyclic hydrocarbon referred to herein containing a total of from 3 to 10 carbon atoms, preferably 5-8 ring carbon atoms. Exemplary cycloalkyls include monocyclic rings having from 3-7, preferably 3-6, carbon atoms, such as cyclopropyl, cyclobutyl, cyclopentyl, cyclohexyl, cycloheptyl and the like. Illustrative examples of cycloalkyl are derived from, but not limited to, the following:

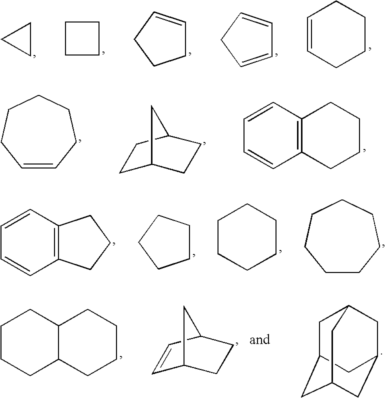

The term “aryl”, as used herein, unless otherwise indicated, includes an organic radical derived from an aromatic hydrocarbon by removal of one hydrogen, such as phenyl or naphthyl.

The term “4-10 membered heterocyclic”, as used herein, unless otherwise indicated, includes aromatic and non-aromatic heterocyclic groups containing one to four heteroatoms each selected from O, S and N, wherein each heterocyclic group has from 4-10 atoms in its ring system, and with the proviso that the ring of said group does not contain two adjacent O or S atoms. Non-aromatic heterocyclic groups include groups having only 4 atoms in their ring system, but aromatic heterocyclic groups must have at least 5 atoms in their ring system. The heterocyclic groups include benzo-fused ring systems. An example of a 4 membered heterocyclic group is azetidinyl (derived from azetidine). An example of a 5 membered heterocyclic group is thiazolyl and an example of a 10 membered heterocyclic group is quinolinyl. Examples of non-aromatic heterocyclic groups are pyrrolidinyl, tetrahydrofuranyl, dihydrofuranyl, tetrahydrothienyl, tetrahydropyranyl, dihydropyranyl, tetrahydrothiopyranyl, piperidino, morpholino, thiomorpholino, thioxanyl, piperazinyl, azetidinyl, oxetanyl, thietanyl, homopiperidinyl, oxepanyl, thiepanyl, oxazepinyl, diazepinyl, thiazepinyl, 1,2,3,6-tetrahydroptridinyl, 2-pyrrolinyl, 3-pyrrolinyl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, 3H-indolyl and quinolizinyl. Examples of aromatic heterocyclic groups are pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl. The foregoing groups, as derived from the groups listed above, may be C-attached or N-attached where such is possible. For instance, a group derived from pyrrole may be pyrrol-1-yl (N-attached) or pyrrol-3-yl (C-attached). Further, a group derived from imidazole may be imidazol-1-yl (N-attached) or imidazol-3-yl (C-attached). The 4-10 membered heterocyclic may be optionally substituted on any ring carbon, sulfur, or nitrogen atom(s) by one to two oxo, per ring. An example of a heterocyclic group wherein 2 ring carbon atoms are substituted with oxo moieties is 1,1-dioxo-thiomorpholinyl. Other Illustrative examples of 4-10 membered heterocyclic are derived from, but not limited to, the following:

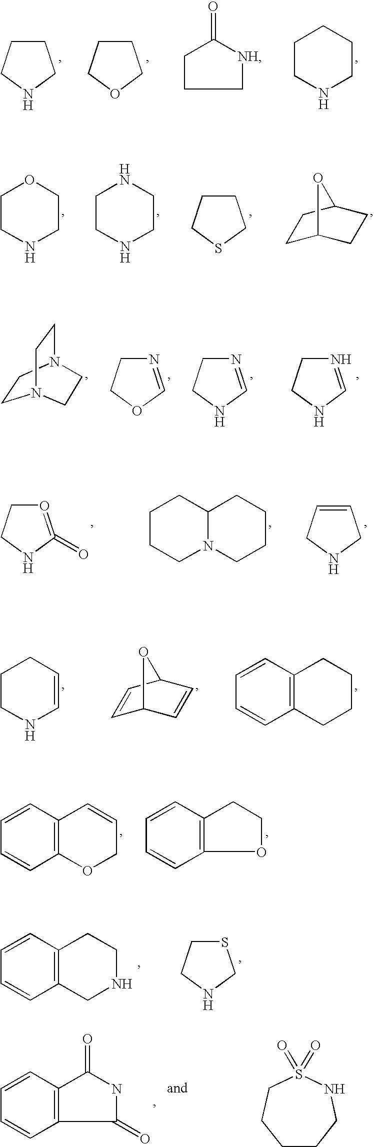

Unless otherwise indicated, the term “oxo” refers to ═O.

The phrase “pharmaceutically acceptable salt(s)”, as used herein, unless otherwise indicated, includes salts of acidic or basic groups which may be present in the compounds of formula 1 or 2. The compounds of formula 1 or 2 that are basic in nature are capable of forming a wide variety of salts with various inorganic and organic acids. The acids that may be used to prepare pharmaceutically acceptable acid addition salts of such basic compounds of formula 1 or 2, are those that form non-toxic acid addition salts, i.e., salts containing pharmacologically acceptable anions, such as the acetate, benzenesulfonate, benzoate, bicarbonate, bisulfate, bitartrate, borate, bromide, calcium edetate, camsylate, carbonate, chloride, clavulanate, citrate, dihydrochloride, edetate, edislyate, estolate, esylate, ethylsuccinate, fumarate, gluceptate, gluconate, glutamate, glycollylarsanilate, hexylresorcinate, hydrabamine, hydrobromide, hydrochloride, iodide, isothionate, lactate, lactobionate, laurate, malate, maleate, mandelate, mesylate, methylsuffate, mucate, napsylate, nitrate, oleate, oxalate, pamoate (embonate), palmitate, pantothenate, phospate/diphosphate, polygalacturonate, salicylate, stearate, subacetate, succinate, tannate, tartrate, teoclate, tosylate, triethiodode, and valerate salts.

In the compounds of formula 1 or 2 where terms such as (CR¹R²)_{q} or (CR¹R²)ₜ are used, R¹ and R² may vary with each iteration of q or t above 1. For instance, where q or t is 2 the terms (CR¹R²)_{q} or (CR¹R²)ₜ may equal —CH₂CH₂—, or —CH(CH₃)C(CH₂CH₃)(CH₂CH₂CH₃)—, or any number of similar moieties falling within the scope of the definitions of R¹ and R². Further, as noted above, any substituents comprising a CH₃(methyl), CH₂(methylene), or CH(methine) group which is not attached to a halogeno, SO or SO₂ group or to a N, O or S atom optionally bears on said group a substituent selected from hydroxy, C₁-C₄ alkoxy and —NR¹R².

Certain compounds of formula 1 or 2, may have asymmetric centers and therefore exist in different enantiomeric forms. All optical isomers and stereoisomers of the compounds of formula 1 or 2 and mixtures thereof, are considered to be within the scope of the invention. With respect to the compounds of formula 1 or 2, the invention includes the use of a racemate, one or more enantiomeric forms, one or more diastereomeric forms, or mixtures thereof. The compounds of formula 1 or 2, may also exist as tautomers. This invention relates to the use of all such tautomers and mixtures thereof.

Certain functional groups contained within the compounds of the present invention can be substituted for bioisosteric groups, that is, groups which have similar spatial or electronic requirements to the parent group, but exhibit differing or improved physicochemical or other properties. Suitable examples are well known to those of skill in the art, and include, but are not limited to moieties described in Patini et al., Chem. Rev, 1996, 96, 3147-3176 and references cited therein.

The subject invention also includes isotopically-labelled compounds, which are identical to those recited in Formula 1, but for the fact that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorous, fluorine and chlorine, such as ²H, ³H, ¹³C, ¹⁴C, ¹⁵N, ¹⁸O, ¹⁷O, ³¹P, ³²P , ³⁵S, ¹⁸F, and ³⁶Cl, respectively. Compounds of the present invention, prodrugs thereof, and pharmaceutically acceptable salts of said compounds or of said prodrugs which contain the aforementioned isotopes and/or other isotopes of other atoms are within the scope of this invention. Certain isotopically-labelled compounds of the present invention, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labelled compounds of Formula 1 or 2 of this invention and prodrugs thereof can generally be prepared by carrying out the procedures disclosed in the Schemes and/or in the Examples and Preparations below, by substituting a readily available isotopically labelled reagent for a non-isotopically labelled reagent.

This invention also encompasses pharmaceutical compositions containing and methods of treating SARS infections through administering prodrugs of compounds of formula 1 or 2 Compounds of formula 1 or 2 having free amino, amido, hydroxy or carboxylic groups can be converted into prodrugs. Prodrugs include compounds wherein an amino acid residue, or a polypeptide chain of two or more (e.g., two, three or four) amino acid residues is covalently joined through an amide or ester bond to a free amino, hydroxy or carboxylic acid group of compounds of formula 1 or 2. The amino acid residues include but are not limited to the 20 naturally occurring amino acids commonly designated by three letter symbols and also includes 4-hydroxyproline, hydroxylysine, demosine, isodemosine, 3-methylhistidine, norvalin, beta-alanine, gamma-aminobutyric acid, citrulline homocysteine, homoserine, ornithine and methionine sulfone. Additional types of prodrugs are also encompassed. For instance, free carboxyl groups can be derivatized as amides or alkyl esters. Free hydroxy groups may be derivatized using groups including but not limited to hemisuccinates, phosphate esters, dimethylaminoacetates, and phosphoryloxymethyloxycarbonyls, as outlined in Advanced Drug Delivery Reviews, 1996, 19, 115. Carbamate prodrugs of hydroxy and amino groups are also included, as are carbonate prodrugs, sulfonate esters and sulfate esters of hydroxy groups. Derivatization of hydroxy groups as (acyloxy)methyl and (acyloxy)ethyl ethers wherein the acyl group may be an alkyl ester, optionally substituted with groups including but not limited to ether, amine and carboxylic acid functionalities, or where the acyl group is an amino acid ester as described above, are also encompassed. Prodrugs of this type are described in J. Med. Chem. 1996, 39, 10. Free amines can also be derivatized as amides, sulfonamides or phosphonamides. All of these prodrug moieties may incorporate groups including but not limited to ether, amine and carboxylic acid functionalities.

The term “SARS-inhibiting agent” means any SARS related coronavirus 3C like protease inhibitor compound represented by formula 1 or a pharmaceutically acceptable salt, hydrate, prodrug, active metabolite or solvate thereof.

The term “interfering with or preventing” SARS-related coronavirus (“SARS”) viral replication in a cell means to reduce SARS replication or production of SARS components necessary for progeny virus in a cell. Simple and convenient assays to determine if SARS viral replication has been reduced include an ELISA assay for the presence, absence, or reduced presence of anti-SARS antibodies in the blood of the subject (Nasoff et al., PNAS 88:5462-5466, 1991), RT-PCR (Yu et al., in Viral Hepatitis and Liver Disease 574-477, Nishioka, Suzuki and Mishiro (Eds.); Springer-Verlag Tokyo, 1994). Such methods are well known to those of ordinary skill in the art. Alternatively, total RNA from transduced and infected “control” cells can be isolated and subjected to analysis by dot blot or northern blot and probed with SARS specific DNA to determine if SARS replication is reduced. Alternatively, reduction of SARS protein expression can also be used as an indicator of inhibition of SARS replication. A greater than fifty percent reduction in SARS replication as compared to control. cells typically quantitates a prevention of SARS replication.

If an inhibitor compound used in the method of the invention is a base, a desired salt may be prepared by any suitable method known to the art, including treatment of the free base with an inorganic acid (such as hydrochloric acid, hydrobromic acid, sulfuric acid, nitric acid, phosphoric acid, and the like), or with an organic acid (such as acetic acid, maleic acid, succinic acid, mandelic acid, fumaric acid, malonic acid, pyruvic acid, oxalic acid, glycolic acid, salicylic acid, pyranosidyl acid (such as glucuronic acid or galacturonic acid), alpha-hydroxy acid (such as citric acid or tartaric acid), amino acid (such as aspartic acid or glutamic acid), aromatic acid (such as benzoic acid or cinnamic acid), sulfonic acid (such as p-toluenesulfonic acid or ethanesulfonic acid), and the like.

If an inhibitor compound used in the method of the invention is an acid, a desired salt may be prepared by any suitable method known to the art, including treatment of the free acid with an inorganic or organic base (such as an amine (primary, secondary, or tertiary)), an alkali metal hydroxide, or alkaline earth metal hydroxide. Illustrative examples of suitable salts include organic salts derived from amino acids (such as glycine and arginine), ammonia, primary amines, secondary amines, tertiary amines, and cyclic amines (such as piperidine, morpholine, and piperazine), as well as inorganic salts derived from sodium, calcium, potassium, magnesium, manganese, iron, copper, zinc, aluminum, and lithium.

In the case of inhibitor compounds, prodrugs, salts, or solvates that are solids, it is understood by those skilled in the art that the hydroxamate compound, prodrugs, salts, and solvates used in the method of the invention, may exist in different polymorph or crystal forms, all of which are intended to be within the scope of the present invention and specified formulas. In addition, the hydroxamate compound, salts, prodrugs and solvates used in the method of the invention may exist as tautomers, all of which are intended to be within the broad scope of the present invention.

As generally understood by those skilled in the art, an optically pure compound is one that is enantiomerically pure. As used herein, the term “optically pure” is intended to mean a compound comprising at least a sufficient activity. Preferably, an optically pure amount of a single enantiomer to yield a compound having the desired pharmacological pure compound of the invention comprises at least 90% of a single isomer (80% enantiomeric excess), more preferably at least 95% (90% e.e.), even more preferably at least 97.5% (95% e.e.), and most preferably at least 99% (98% e.e.).

The term “treating”, as used herein, unless otherwise indicated, means reversing, alleviating, inhibiting the progress of, or preventing the disorder or condition to which such term applies, or one or more symptoms of such disorder or condition. The term “treatment”, as used herein, unless otherwise indicated, refers to the act of treating as “treating” is defined immediately above. In a preferred embodiment of the present invention, “treating” or “treatment” means at least the mitigation of a disease condition in a human, that is alleviated by the inhibition of the activity of one or more coronaviral 3C-like proteases, including, but not limited to the 3C-like protease of the causative agent for SARS. In the case of SARS, representative disease conditions include fever, dry cough, dyspnea, headache, hypoxemia, lymphopenia, elevated aminotransferase levels as well as viral titer. Methods of treatment for mitigation of a disease condition include the use of one or more of the compounds in the invention in any conventionally acceptable manner. According to certain preferred embodiments of the invention, the compound or compounds of the present invention are administered to a mammal, such as a human, in need thereof. Preferably, the mammal in need thereof is infected with a coronavirus such as the causative agent of SARS.

The present invention also includes prophylactic methods, comprising administering an effective amount of a compound of the invention, or a pharmaceutically acceptable salt, prodrug, pharmaceutically active metabolite, or solvate thereof to a mammal, such as a human, at risk for infection by a coronavirus. According to certain preferred embodiments, an effective amount of one or more compounds of the invention, or a pharmaceutically acceptable salt, prodrug, pharmaceutically active metabolite, or solvate thereof is administered to a human at risk for infection by the causative agent for SARS. The prophylactic methods of the invention include the use of one or more of the compounds in the invention in any conventionally acceptable manner.

Recent evidence indicates that a new coronavirus is the causative agent of SARS. The nucleotide sequence of the SARS-associated coronavirus has also recently been determined and made publicly available.

The activity of the inhibitor compounds as inhibitors of SARS-related viral activity may be measured by any of the suitable methods available in the art, including in vivo and in vitro assays. The activity of the compounds of the present invention as inhibitors of coronavirus 3C-like protease activity (such as the 3C-like protease of the SARS coronavirus) may be measured by any of the suitable methods known to those skilled in the art, including in vivo and in vitro assays. Examples of suitable assays for activity measurements include the antiviral cell culture assays described herein as well as the antiprotease assays described herein, such as the assays described in Examples 1 through 3.

Administration of the inhibitor compounds and their pharmaceutically acceptable prodrugs, salts, active metabolites, and solvates may be performed according to any of the accepted modes of administration available to those skilled in the art. Illustrative Examples of suitable modes of administration include oral, nasal, pulmonary, parenteral, topical, transdermal, and rectal. Oral, intravenous, and nasal deliveries are preferred.

A SARS-inhibiting agent may be administered as a pharmaceutical composition in any suitable pharmaceutical form. Suitable pharmaceutical forms include solid, semisolid, liquid, or lyopholized formulations, such as tablets, powders, capsules, suppositories, suspensions, liposomes, and aerosols. The SARS-inhibiting agent may be prepared as a solution using any of a variety of methodologies. For example, the SARS-inhibiting agent can be dissolved with acid (e.g., 1 M HCl) and diluted with a sufficient volume of a solution of 5% dextrose in water (D5W) to yield the desired final concentration of SARS-inhibiting agent (e.g., about 15 mM). Alternatively, a solution of D5W containing about 15 mM HCl can be used to provide a solution of the SARS-inhibiting agent at the appropriate concentration. Further, the SARS-inhibiting agent can be prepared as a suspension using, for example, a 1% solution of carboxymethylcellulose (CMC).

Acceptable methods of preparing suitable pharmaceutical forms of the pharmaceutical compositions are known or may be routinely determined by those skilled in the art. For example, pharmaceutical preparations may be prepared following conventional techniques of the pharmaceutical chemist involving steps such as mixing, granulating, and compressing when necessary for tablet forms, or mixing, filling, and dissolving the ingredients as appropriate, to give the desired products for oral, parenteral, topical, intravaginal, intranasal, intrabronchial, intraocular, intraaural, and/or rectal administration.

Pharmaceutical compositions of the invention may also include suitable excipients, diluents, vehicles, and carriers, as well as other pharmaceutically active agents, depending upon the intended use. Solid or liquid pharmaceutically acceptable carriers, diluents, vehicles, or excipients may be employed in the pharmaceutical compositions. Illustrative solid carriers include starch, lactose, calcium sulfate dihydrate, terra alba, sucrose, talc, gelatin, pectin, acacia, magnesium stearate, and stearic acid. Illustrative liquid carriers include syrup, peanut oil, olive oil, saline solution, and water. The carrier or diluent may include a suitable prolonged-release material, such as glyceryl monostearate or glyceryl distearate, alone or with a wax. When a liquid carrier is used, the preparation may be in the form of a syrup, elixir, emulsion, soft gelatin capsule, sterile injectable liquid (e.g., solution), or a nonaqueous or aqueous liquid suspension.

A dose of the pharmaceutical composition may contain at least a therapeutically effective amount of an SARS-inhibiting agent and preferably is made up of one or more pharmaceutical dosage units. The selected dose may be administered to a mammal, for example, a human patient, in need of treatment mediated by inhibition of SARS-related coronavirus activity, by any known or suitable method of administering the dose, including topically, for example, as an ointment or cream; orally; rectally, for example, as a suppository; parenterally by injection; intravenously; or continuously by intravaginal, intranasal, intrabronchial, intraaural, or intraocular infusion. When the composition is administered in conjunction with a cytotoxic drug, the composition can be administered before, with, and/or after introduction of the cytotoxic drug. However, when the composition is administered in conjunction with radiotherapy, the composition is preferably introduced before radiotherapy is commenced.

The phrases “therapeutically effective amount” and “effective amount” are intended to mean the amount of an inventive agent that, when administered to a mammal in need of treatment, is sufficient to effect treatment for injury or disease conditions alleviated by the inhibition of SARS viral replication such as for potentiation of anti-cancer therapies or inhibition of neurotoxicity consequent to stroke, head trauma, and neurodegenerative diseases. The amount of a given SARS-inihibiting agent used in the method of the invention that will be therapeutically effective will vary depending upon factors such as the particular SARS-inihibiting agent, the disease condition and the severity thereof, the identity and characteristics of the mammal in need thereof, which amount may be routinely determined by artisans.

It will be appreciated that the actual dosages of the SARS-inhibiting agents used in the pharmaceutical compositions of this invention will be selected according to the properties of the particular agent being used, the particular composition formulated, the mode of administration and the particular site, and the host and condition being treated. Optimal dosages for a given set of conditions can be ascertained by those skilled in the art using conventional dosage-determination tests. For oral administration, e.g., a dose that may be employed is from about 0.001 to about 1000 mg/kg body weight, preferably from about 0.1 to about 100 mg/kg body weight, and even more preferably from about 1 to about 50 mg/kg body weight, with courses of treatment repeated at appropriate intervals.

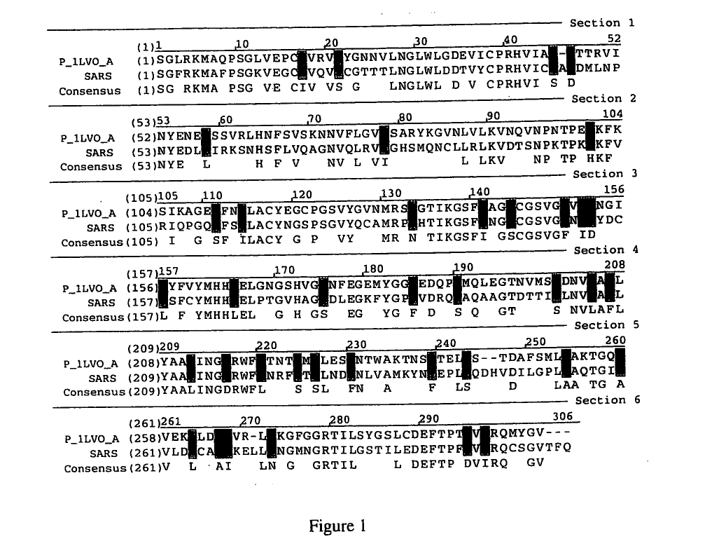

Protein functions required for coronavirus replication and transcription are encoded by the so-called “replicase” gene. Two overlapping polyproteins are translated from this gene and extensively processed by viral proteases. The C-proximal region is processed at eleven conserved interdomain junctions by the coronavirus main or “3C-like” protease. The name “3C-like” protease derives from certain similarities between the coronavirus enzyme and the well-known picornavirus 3C proteases. These include substrate preferences, use of cysteine as an active site nucleophile in catalysis, and similarities in their putative overall polypeptide folds. A comparison of the amino acid sequence of the SARS-associated coronavirus 3C-like protease to that of other known coronaviruses shows the amino acid sequence to be highly conserved, particularly in the catalytically important regions of the protease (FIG. 1).

Amino acids of the substrate in the protease cleavage site are numbered from the N to the C terminus as follows: -P3-P2-P1-P1′-P2′-P3′, with cleavage occurring between the P1 and P1′ residues (Schechter & Berger, 1967). Substrate specificity is largely determined by the P2, P1 and P1′ positions. Coronavirus main protease cleavage site specificities are highly conserved with a requirement for glutamine at P1 and a small amino acid at P1′ (Journal of General Virology 83, pp. 595-599 (2002)).

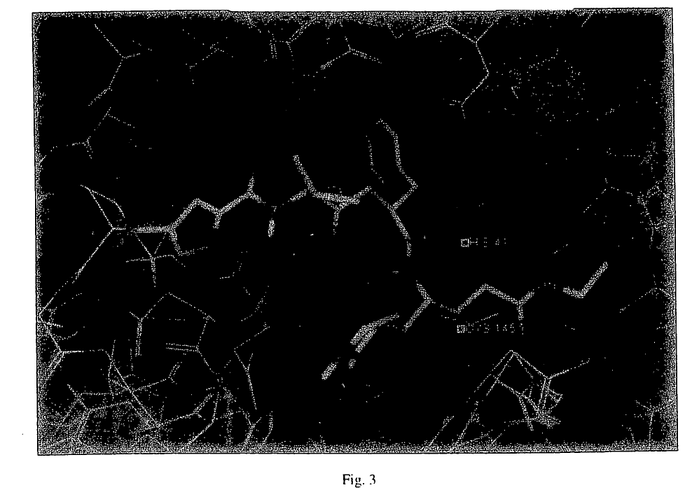

Recently, Hilgenfeld and colleagues published a high-resolution x-ray structure of the porcine transmissible gastroenteritis coronavirus main protease (The EMBO Journal, Vol. 21, pp. 3213-3224 (2002)). Atomic coordinates are available through the Protein Data Bank under accession code 1LVO. Our observations of the catalytic and structural similarities between rhinovirus 3C protease and coronavirus “3C-like” main protease, lead to the conclusion that selected inhibitors of rhinovirus 3C protease would be useful against the coronavirus main (3C-like) protease (FIG. 3).

Several considerations come into play when developing strategies for design of therapeutically efficacious serine and cysteine protease inhibitors. For many of these proteins, specificity pockets for substrate (or inhibitor) recognition are shallow, and binding determinants are widely dispersed over large surface areas. Difficulties inherent in discovering small molecules with high affinity for such binding sites are in many respects analogous to those encountered in attempting to disrupt proteinBprotein interactionswith small effector molecules. Serine proteases such as factor Xa and thrombin, proteins involved in the blood-coagulation pathway with deep well defined S1 specificity pockets, have been targeted effectively with structurally diverse, small, noncovalent inhibitors and thus are exceptions to this generalization. However, for virally encoded serine and cysteine proteases of known structure, such as the herpes family of serine proteases, hepatitis C NS3 protease, picornavirus 3C proteases and coronaviral 3C-like proteases, the fact that substrate recognition is modulated by extensive proteinBprotein interactions represents a significant impediment for design of specific inhibitors.

Peptidic substrates in which the scissile amide carbonyl is replaced by a Michael acceptor were first introduced as specific irreversible inhibitors of the cysteine protease papain by Hanzlik and coworkers. We reasoned that, although this reaction is probably facilitated by the especially nucleophilic thiolateimidazolium ion pair in papain-like cysteine proteases, suitably activated Michael acceptors might also undergo addition by the presumably less nucleophilic catalytic cysteine of 3C and 3C-like proteases.

Covalent irreversible inactivation of 3C and 3C-like proteases by Michael acceptors proceeds according to a kinetic mechanism that can be broken down into two parts.

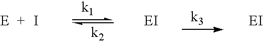

The inhibitor initially forms a reversible encounter complex with 3C, which can then undergo a chemical step (nucleophilic attack by the reactive site Cys) leading to stable covalent-bond formation. The observed second-order rate constant for inactivation (k_{obs}/l) depends on both the equilibrium binding constant k₂/k₁ and the chemical rate for covalent bond formation k₃ (Meara, J. P. & Rich, D. H. (1995) Bioorg. Med. Chem. Lett. 5, 2277-2282). We anticipated that Michael-acceptor inhibitors with specificity for 3C-like protease, as with 3C protease, would likely achieve high rates of enzyme inactivation by combining good equilibrium binding with a modest rate of covalent-bond formation. The rate of chemical inactivation presumably depends on not only the intrinsic electrophilic character of the inhibitor, but on how the reactive vinyl group is oriented relative to the Cys in the reactive site before nucleophilic attack and on the extent to which the transition state for the reaction can be stabilized by the enzyme. Mechanism-based activation of an inherently weak Michael acceptor as a means of increasing the rate of the chemical step, and thus k_{obs}/l, is conceptually more attractive than attempting to achieve a similar effect by simply increasing intrinsic electrophilic reactivity, which would likely impart undesirable properties to such compounds.

### EXAMPLES

In the examples described below, unless otherwise indicated, all temperatures are set forth in degrees Celsius and all parts and percentages are by weight. Reagents may be purchased from commercial suppliers, such as Sigma-Aldrich Chemical Company, or Lancaster Synthesis Ltd. and may be used without further purification unless otherwise indicated. Tetrahydrofuran (THF) and N,N-dimethylformamide (DMF) may be purchased from Aldrich in Sure Seal bottles and used as received. All solvents may be purified using standard methods known to those skilled in the art, unless otherwise indicated.

Preferred compounds in accordance with the invention may be prepared in manners analogous to those specifically described below.

### Example 1

### Protection from Infection

The ability of compounds to protect cells against infection by the SARS coronavirus is measured by a cell viability assay similar to that described in Weislow, O. S., Kiser, R., Fine, D. L., Bader, J., Shoemaker, R. H., and Boyd, M. R. 1989. New Soluble-Formazan Assay for HIV-1 Cytopathic Effects: Application to High-Flux Screening of Synthetic and Natural Products for AIDS-Antiviral Activity. Journal of the National Cancer Institute 81(08):577-586), utilizing neutral red staining as an endpoint. Briefly, Vero cells are resuspended in medium containing appropriate concentrations of compound or medium only. Cells are infected with SARS-associated virus or mock-infected with medium only. One to seven days later, neutral red is added to the test plates and following incubation at 37° C. for one hour, cells are solubilized and the amount of neutral red produced is quantified spectrophotometrically at 540 nm. Data is expressed as the percent of neutral red produced in compound-treated cells compared to neutral red produced in wells of uninfected, compound-free cells. The fifty percent effective concentration (EC50) is calculated as the concentration of compound that increases the percent of neutral red production in infected, compound-treated cells to 50% of that produced by uninfected, compound-free cells. The 50% cytotoxicity concentration (CC50) is calculated as the concentration of compound that decreases the percentage of neutral red produced in uninfected, compound-treated cells to 50% of that produced in uninfected, compound-free cells. The therapeutic index is calculated by dividing the cytotoxicity (CC50) by the antiviral activity (EC50).

### Example 2

### Viral Yield Assay

The ability of compounds to protect cells by infection is evaluated in a virus yield assay similar to that described in A. K. Patick, S. L. Binford, M. A. Brothers, R. L. Jackson, C. E. Ford, M. D. Diem, F. Maldonado, P. S. Dragovich, R. Zhou, T. J. Prins, S. A. Fuhrman, J. W. Meador, L. S. Zalman, D. A. Matthews and S. T. Worland. 1999. In vitro antiviral activity of AG7088, a potent inhibitor of human rhinovirus 3C protease. Antimicrob. Agents and Chemo. 43:2444-2450. Briefly, 0.2 ml of serial ten-fold dilutions of SARS-associated virus is allowed to adsorb onto monolayers of Vero cells. After one hour adsorption, the cell monolayers are washed twice with PBS and overlayed with medium containing 0.5% Seaplaque agarose (FMC Bioproducts, Rockland, Me). After one to seven days of incubation at 34° C., the cell monolayers are fixed with EAF (65% ethanol, 22% acetic acid, and 4% formaldehyde), stained with 1% crystal violet and virus plaques enumerated. Data is expressed as plaque forming units (PFU) per ml. The fifty percent EC50 is calculated as the concentration of compound that decreases the number of PFU/ml in infected, compound-treated cells to 50% of that produced by infected, compound-free cells.

### Example 3

### Coronavirus 3C Protease FRET Assay and Analysis

Proteolytic activity of Coronavirus 3C protease is measured using a continuous fluorescence resonance energy transfer assay. The substrate, DABCYL-GRAVFQGPVG-EDANS, is prepared by modification of the core decapeptide (American Peptide Systems) and purified prior to use by HPLC using a C-18 resin (Alltech). Other peptide cores are possible and may, for example, be derived from protease cleavage sites in the published sequence of the SARS coronavirus. Preferred peptides retain the P1 and P1′ amino acids (QG) of the above decapeptide (the proteolytic cleavage site). In addition, other fluorescent probe/quencher combinations are possible. The assays include reaction buffer (50 mM Tris, pH 7.5, 1 mM EDTA 0.1 to 10 μM substrate, 5 to 50 nM coronavirus 3C protease, 2% DMSO and inhibitor as appropriate. Cleavage of the DABCYL-EDANS substrate peptide is monitored by the appearance of fluorescent emission at 490 nm (following excitation at 336 nm). Data are analyzed with the non-linear regresssion analysis program Kalidagraph using the equation:
FU=offset+(limit)(1−e^{-(kobs)t})
where offset equals the fluorescence signal of the uncleaved peptide substrate, and limit equals the fluorescence of fully cleaved peptide substrate. The kobs is the first order rate constant for this reaction, and in the absence of any inhibitor represents the utilization of substrate. In an enzyme start reaction which contains an irreversible inhibitors, and where the calculated limit is less than 20% of the theoretical maximum limit, the calculated kobs represents the rate of inactivation of coronavirus 3C protease. The slope (kobs/l) of a plot of kobs vs. [l] is a measure of the avidity of the inhibitor for an enzyme. For very fast irreversible inhibitors, kobs/l is calculated from observations at only one or two [l] rather than as a slope.

### Example 4

### Structure-Assisted Selection of Michael Acceptor-Based Inhibitors of 3C-like Protease Inhibitors

Homology Modeling

A homology model for SARS 3C-like protease was created using the atomic coordinates for the recently published coronavirus “3C-like” protease as a template. BLAST was employed to identify the 3C-like proteinase from the genomic RNA sequence of SARS (AY274119). Minor adjustment to the BLAST output resulted in an alignment with high percent identity and few gaps (FIG. 1), and this alignment was used to create a homology model with the MODELLER package in Insight2000 (Sanchez and Sali 2000).

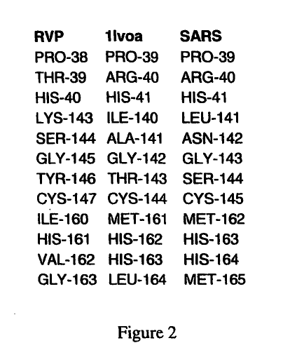

Twelve residues with high structural conservation (FIG. 2) were identified by visual inspection of the rhinovirus 3C (1CQQ) and TGEV 3C-like proteinase (1LVO) structures, as well as the SARS 3C-like proteinase homology model. The structures were superimposed in a common reference frame by minimizing the root mean square difference (RMSD) between the backbone atoms of these residues, with RMSD<0.6 Angstroms². Inspection of the structures in the common reference frame demonstrates strong conservation of the side-chain conformations of the catalytic cysteine and histidine residues (FIG. 3).

The compounds of Examples 8-15 were modeled into the SARS 3CL protease structure by fixing the lactam side chain and ester Michael acceptor in the orientation found in the cocrystal structure of AG7088 bound to RVP 3C protease (PDB accession code 1CQQ), and optimizing the binding of the remaining portion of the ligand using an automated docking application (D K Gehlhaar, G M Verkhivker, P A Rejto, C J Sherman, D B Fogel, L J Fogel, S T Freer, Chem. Biol. 2, 317-324 (1995)). The structure of the SARS 3CL protease was obtained from an apo structure (PDB accession code 1Q2W) and was prepared for modeling by completing missing side chains and optimizing the positions of polar hydrogens.

Modeling Analysis

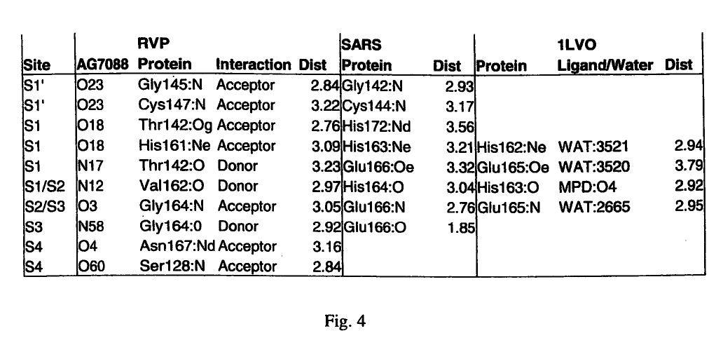

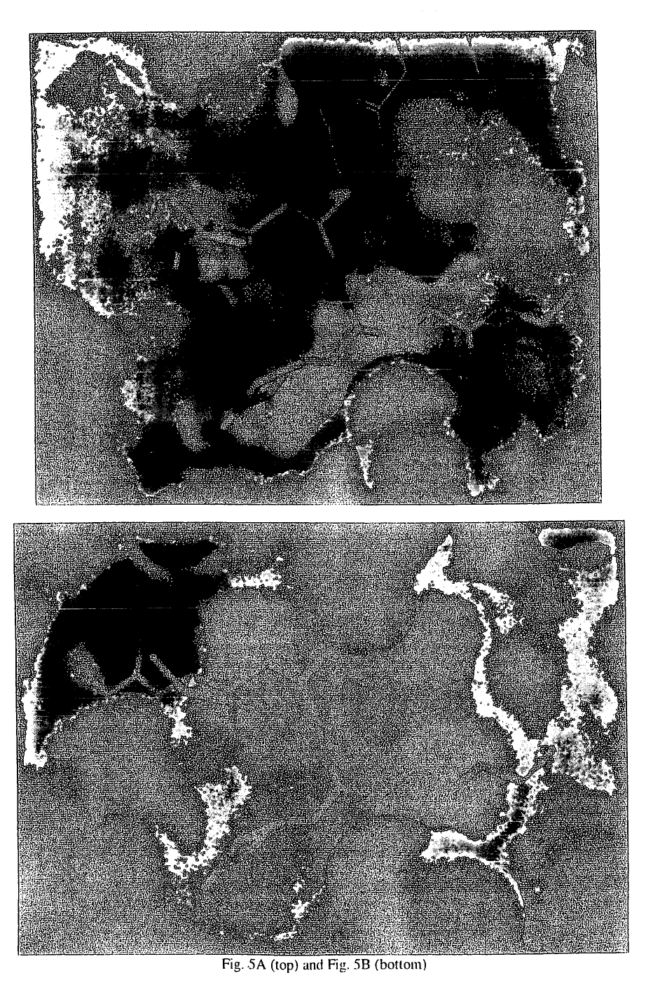

Electronic and steric characteristics of coronavirus “3C-like” protease near the active site cysteine and the adjacent S1 and S1′ specificity pockets are similar to those of rhinovirus 3C protease with corresponding features closely aligned based on the structural superposition described above. In the S1′ specificity pocket, main-chain nitrogens Gly145 and Cys147 activate the carbonyl oxygen in AG7088. The sequence and structural location of these two residues are conserved in the TGEV structure (Gly142 and Cys144). In the S1 specificity pocket, there are three hydrogen bonds between AG7088 and rhinovirus 3C protease (FIG. 4). These three hydrogen bonds are preserved in the SARS model, one of them involving a corresponding His Nitrogen in the two proteins, and the others substituted with alternate residues. Despite substitutions in the sequence of the S1 pocket, the solvent accessible surfaces of rhinovirus 3C protease and the SARS model have considerable agreement in the P1 binding site (FIG. 5). Further examination of the superposed structures indicates that an inhibitor such as AG7088 could all seven of the hydrogen bonds with coronavirus “3C-like” protease at P1, P2, and P3 that are observed for rhinovirus 3C protease (FIG. 4). Differences between the structures are most prevalent in the S3 and S4 pockets, suggesting that optimal inhibitors of rhinovirus 3C protease and SARS will differ in this region. Furthermore, the S2 specificity pocket is more constrained in the coronavirus protease, suggesting that inhibitors having side chains smaller than fluorophenyalanine (as in AG7088) could be preferred. This is consistent with the prevalence of Leu in many of the known coronavirus cleavage site sequences (Hegyi and Ziebuhr 2002). Coronavirus main protease cleavage site specificities are highly conserved with a requirement for glutamine at P1 and a small amino acid at P1′ (Hegyi and Ziebuhr 2002). Picornavirus 3C proteases also favor cleavage sites with glutamine at P1 and either Gly or Ala at P1′. The structural superposition described above indicates that the two proteins differ considerably in exactly how their respective S4 specificity pockets are constructed. The polypeptide chain loops that form S4 are also positioned differently relative to S1, S2, and S3 in the two viral proteases.

The modeling analysis leads to the following suggestions for inhibitors:
1. Michael acceptor based inhibitors with appropriate specificity elements should covalently inactivate coronavirus “3C-like” protease with both methyl and ethyl ester containing compounds.
2. Compounds with glutamine or the lactam side chain at P1 should be chosen.
3. Compounds with differing substituents at P2 should be selected including phe but also smaller side chains such as leu and val.
4. Wide variability should be acceptable at P3 as this side chain site is fully solvent accessible.
5. Size and conformational flexibility at P4 may be important. Smaller is probably better than larger based on modeling. Include thiocarbamate containing analogs.

Michael acceptor containing SARS protease inhibitor compounds are selected based on the above qualitative criteria. Alternatively, one may also dock available compounds to a homology model of the SARS protease. Such a model could be constructed using the known structure of porcine coronavirus protease and the gene sequence of the SARS virus “3C-like” protease.

### Example 5

### Michael Acceptor-Based Inhibitors of the SARS Protease

Michael acceptor-based inhibitors having the criteria discussed above are assayed using the protease and antiviral assays described above in Examples 1-3. The following compounds are identified as inhibitors of the 3C-like protease of the SARS-associated virus.

Table 1 below provides examples of inhibitor compounds that are useful as SARS-related 3C protease inhibitors. The examples and preparations provided below further illustrate and exemplify the compounds of the present invention and methods of preparing such compounds. It is to be understood that the scope of the present invention is not limited in any way by the scope of the following examples and preparations. In the following examples molecules with a single chiral center, unless otherwise noted, exist as a racemic mixture. Those molecules with two or more chiral centers, unless otherwise noted, exist as a racemic mixture of diastereomers. Single enantiomers/diastereomers may be obtained by methods known to those skilled in the art.

In the following examples and preparations, “Et” means ethyl, “AC” means acetyl, “Me” means methyl, “ETOAC” or “ETOAc” means ethyl acetate, “THF” means tetrahydrofuran, and “Bu” means butyl.

TABLE 1

| | CHEM NAME | MOL FORMULA | MOL Wt |
|---|---|---|---|
| 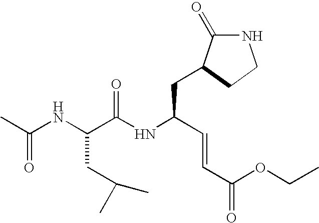 | 4-(2-Acetylamino-4-methyl- pentanoylamino)-5-(2-oxo- pyrrolidin-3-yl)-pent-2-enoic acid ethyl ester | C₁₉H₃₁N₃O₅ | 381.47 |
| 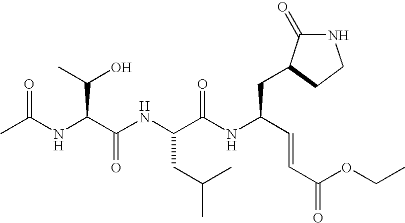 | 4-[2-(2-Acetylamino-3-hydroxy- butyrylamino)-4-methyl- pentanoylamino]-5-(2-oxo- pyrrolidin-3-yl)-pent-2-enoic acid ethyl ester | C₂₃H₃₈N₄O₇ | 482.57 |
| 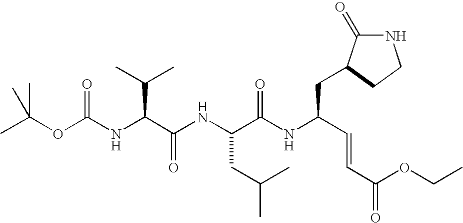 | 4-[2-(2-tert- Butoxycarbonylamino-3-methyl- butyrylamino)-4-methyl- pentanoylamino]-5-(2-oxo- pyrrolidin-3-yl)-pent-2-enoic acid ethyl ester | C₂₇H₄₈N₄O₇ | 538.68 |
| 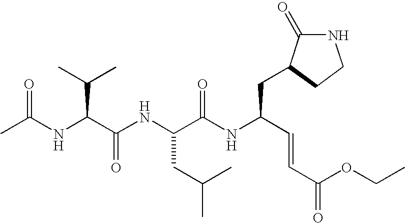 | 4-[2-(2-Acetylamino-3-methyl- butyrylamino)-4-methyl- pentanoylamino]-5-(2-oxo- pyrrolidin-3-yl)-pent-2-enoic acid ethyl ester | C₂₄H₄₀N₄O₆ | 480.60 |
| 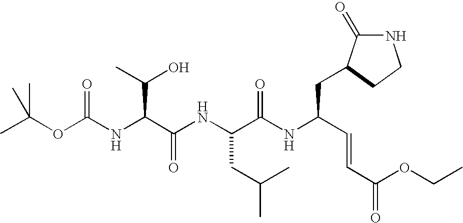 | 4-[2-(2-tert- Butoxycarbonylamino-3-hydroxy- butyrylamino)-4-methyl- pentanoylamino]-5-(2-oxo- pyrrolidin-3-yl)-pent-2-enoic acid ethyl ester | C₂₆H₄₄N₄O₈ | 540.65 |
| 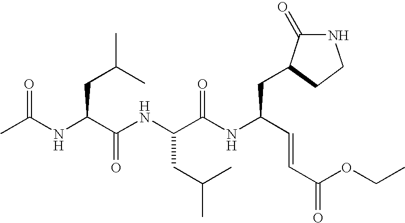 | 4-[2-Acetylamino-4-methyl- pentanoylamino)-4-methyl- pentanoylamino]-5-(2-oxo- pyrrolidin-3-yl)-pent-2-enoic acid ethyl ester | C₂₅H₄₂N₄O₆ | 494.62 |
| 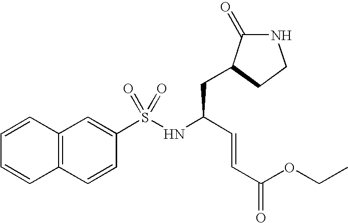 | Ethyl (2E,4S)-4-[(2- naphthylsulfonyl)amino]-5-[(3S)- 2-oxopyrrolidin-3-yl]pent-2- enoate | C₂₁H₂₄N₂O₅S | 416.496 |
| 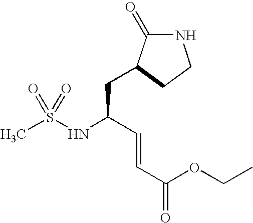 | Ethyl (2E,4S)-4- [(methylsulfonyl)amino]-5-[(3S)- 2-oxopyrrolidin-3-yl]pent-2- enoate | C₁₂H₂₀N₂O₅S | 304.365 |

### Example 5

### Preparation of 4-(2-tert-Butoxycarbonylamino-4-methyl-pentanoylamino)-5-(2-oxo-pyrrolidin-3-yl)-pent-2-enoic acid ethyl ester

To a solution of 4-tert-butoxycarbonylamino-5-(2-oxo-pyrrolidin-3-yl)-pent-2-enoic acid ethyl ester (2.0 g, 6.1 mmol) in 10 mL dioxane was added 10 mL of 4M HCl in dioxane. After 2 hours, concentration of the solution gave a white solid. This was taken up in 10 mL dimethylformamide (DMF). BOC L-leucine (1.68 g, 1.1 equiv) and triethylamine (2.14 mL, 2.5 equiv) was added followed by O-(7-azabenzotriazol-1-yl)-N,N,N′,N′-tetramethyluronium hexafluorophosphate (HATU) (2.8 g, 7.4 mmol) at 0° C. and stirred for 30 minutes after which LC-MS analysis showed that the reaction was completed. The solution was allowed to stir and come to room temperature over 2 hours. Water was added and the product extracted into ethyl acetate (3×) and then concentrated. Purification by flash chromatography (50% EtOAc/hexane-4:1 EtOAc/hexane) gave 2.4 grams (5.4 mmol, 88 %).

### Example 6

### Preparation of 4-(2-Amino-4-methyl-pentanoylamino)-5-(2-oxo-pyrrolidin-3-yl)-pent-2-enoic acid ethyl ester hydrochloride

To a suspension of 4-(2-tert-butoxycarbonylamino-4-methyl-pentanoylamino)-5-(2-oxo-pyrrolidin-3-yl)-pent-2-enoic acid ethyl ester (2.4 g, 5.5 mmol) in 20 mL of dioxane was added, dropwise, 20 mL of 4M HCl in dioxane. The mixture was stirred at room temperature for 4 hours. LC-MS and TLC analysis showed that the starting material had been consumed. The solution was concentrated and dried under vacuum overnight to yield 2.05 g (5.4 mmol, 98%) of a white solid.

### Example 7

### Preparation of Protease Inhibitors

Michael acceptor-based inhibitors are prepared according to the following general method. A solution is prepared containing 4-(2-amino-4-methyl-pentanoylamino)-5-(2-oxo-pyrrolidin-3-yl)-pent-2-enoic acid ethyl ester hydrochloride (1 equiv), the appropriate carboxylic acid or acid chloride (1.1 equiv), triethylamine (2.5 equiv) and HATU (1.2 equiv) in DMF. The solution is stirred at 0° C. for 30 minutes. Complete reaction is confirmed by LC-MS. Water is then added, the product extracted into ethyl acetate (3×), dried over Na₂SO₄ and concentrated. The product is then purified by flash chromatography (2% MeOH/1:1 CHCl₃:EtOAc-1% MeOH/CHCl₃).

### Example 8

### Preparation of 4-[2-(2-tert-Butoxycarbonylamino-3-hydroxy-butyrylamino)-4-methyl-pentanoylamino]-5-(2-oxo-pyrrolidin-3-yl)-pent-2-enoic acid ethyl ester

The method of Example 7 was performed with BOC-Thr-OH as the carboxylic acid to yield 100 mg of 4-[2-(2-tert-Butoxycarbonylamino-3-hydroxy-butyrylamino)-4-methyl-pentanoylamino]-5-(2-oxo-pyrrolidin-3-yl)-pent-2-enoic acid ethyl ester as a white solid. The NMR was as follows: ¹H NMR (400 MHz, CDCl₃) □ 7.91(d, 1H, J=7.83 Hz), 7.05(d, 1H, J=8.58 Hz), 6.80 (dd, 1H, J=5.31, 15.66 Hz), 6.63 (s, 1H), 5.90 (dd, 1H, J=1.52, 15.67 Hz), 5.59 (d, 1H, J=7.83 Hz), 4.55 (bs, 3H), 4.25-4.08 (m, 3H), 3.38-3.29 (m, 2H), 2.42 (s, 2H), 2.02-1.95 (m, 1H), 1.82-1.49 (m, 6H), 1.42 (s, 9H), 1.29-1.21 (m, 3H), 1.14 (d, 3H, J=6.07 Hz), 0.93-0.86 (m, 6H).

### Example 9

### Preparation of 4-[2-(2-tert-Butoxycarbonylamino-3-methyl-butyrylamino)-4-methyl-pentanoylamino]-5-(2-oxo-pyrrolidin-3-yl)-pent-2-enoic acid ethyl ester

The method of Example 7 was performed with BOC-Val-OH as the carboxylic acid to yield 118.5 mg of 4-[2-(2-tert-Butoxycarbonylamino-3-methyl-butyrylamino)-4-methyl-pentanoylamino]-5-(2-oxo-pyrrolidin-3-yl)-pent-2-enoic acid ethyl ester as a white solid. The NMR was as follows: ¹H NMR (300 MHz, CDCl₃) □ 7.58 (d, 1H, J=7.54 Hz), 7.18 (d, 1H, J=8.29 Hz), 6.92 (s, 1H), 6.83 (dd, 1H, J=5.27, 15.82 Hz), 5.90 (d, 1H, J=15.82 Hz), 5.21 (d, 1H, J=8.48 Hz), 4.69-4.64 (m, 2H), 4.22-4.07 (m, 2H), 3.84-3.77 (m, 1H), 3.41-3.24 (m, 2H), 2.35 (bs, 2H), 2.11-1.96 (m, 1H), 1.85-1.72 (m, 1H), 1.69-1.49 (m, 5H), 1.41 (s, 9H), 1.30-1.19 (m, 3H), 0.93-0.86 (m, 12H).

### Example 10

### Preparation of 4-(2-Acetylamino-4-methyl-pentanoylamino)-5-(2-oxo-pyrrolidin-3-yl)-pent-2-enoic acid ethyl ester

The method of Example 7 was performed with Acetyl chloride as the acid chloride to yield 55.3 mg of 4-(2-Acetylamino-4-methyl-pentanoylamino)-5-(2-oxo-pyrrolidin-3-yl)-pent-2-enoic acid ethyl ester as a white solid. The NMR was as follows: ¹H NMR (400 MHz, CDCl₃) □ 8.14 (d, 1H, J=7.07 Hz), 7.11 (s, 1H), 6.81-6.70 (m, 2H), 5.88 (dd, 1H, J=1.26, 14.40 Hz), 4.75-4.69 (m, 1H), 4.46 (bs, 1H), 4.18-4.09 (m, 2H), 3.35-3.24 (m, 2H), 2.40-2.23 (m, 2H), 2.16-2.05 (m, 1H), 1.95 (s, 3H), 1.75-1.46 (m, 5H), 1.28-1.18 (m, 3H), 0.93-0.83 (m, 6H).

### Example 11

### Preparation of 4-[2-(2-Acetylamino-3-hydroxy-butyrylamino)-4-methyl-pentanoylamino]-5-(2-oxo-pyrrolidin-3-yl)-pent-2-enoic acid ethyl ester

The method of Example 7 was performed with AC-Thr-OH as the carboxylic acid to yield 43.1 mg of 4-[2-(2-Acetylamino-3-hydroxy-butyrylamino)-4-methyl-pentanoylamino]-5-(2-oxo-pyrrolidin-3-yl)-pent-2-enoic acid ethyl ester as a white solid. The NMR was as follows: ¹H NMR (300 MHz, CDCl₃) □ 8.01 (d, 1H, J=7.91 Hz), 7.24 (d, 1H, J=7.35 Hz), 6.80 (dd, 1H, J=5.46, 15.64 Hz), 6.71 (d, 1H, J=7.72 Hz), 6.35 (s, 1H), 5.92 (dd, 1H, J=1.50, 15.82 Hz), 4.83 (bs, 1H), 4.64-4.60 (m, 3H), 4.24-4.05 (m, 2H), 3.39-3.32 (m, 2H), 2.49-2.40 (m, 2H), 2.01 (s, 3H), 1.87-1.49 (m, 7H), 1.29-1.21 (m, 3H), 1.15 (d, 3H, J=6.40 Hz), 0.97-0.85 (m, 6H).

### Example 12

### Preparation of 4-[2-(2-Acetylamino-3-methyl-butyrylamino)-4-methyl-pentanoylamino]-5-(2-oxo-pyrrolidin-3-yl)-pent-2-enoic acid ethyl ester

The method of Example 7 was performed with AC-Val-OH as the carboxylic acid to yield 76.8 mg of 4-[2-(2-Acetylamino-3-methyl-butyrylamino)-4-methyl-pentanoylamino]-5-(2-oxo-pyrrolidin-3-yl)-pent-2-enoic acid ethyl ester as a white solid. The NMR was as follows: ¹H NMR (400 MHz, CD3OD) □ 6.31 (dd, 1H, J=5.31, 15.66 Hz), 5.85 (dd, 1H, J=1.77, 14.15 Hz), 4.61-4.52 (m, 1H), 4.33-4.25 (m, 1H), 4.15-4.05 (m, 3H), 3.21-3.11 (m, 1H), 2.51-2.42 (m, 1H), 2.26-2.19 (m, 1H), 2.02-1.95 (m, 2H), 1.93 (s, 3H), 1.78-1.46 (m, 6H), 1.26-1.18 (m, 3H), 0.95-0.84 (m, 12H).

### Example 13

### Preparation of 4-[2-Acetylamino-4-methyl-pentanoylamino)-4-methyl-pentanoylamino]-5-(2-oxo-pyrrolidin-3-yl)-pent-2-enoic acid ethyl ester

The method of Example 7 was performed with AC-Leu-OH as the carboxylic acid to yield 72.8 mg of 4-[2-Acetylamino-4-methyl-pentanoylamino)-4-methyl-pentanoylamino]-5-(2-oxo-pyrrolidin-3-yl)-pent-2-enoic acid ethyl ester as a white solid. The NMR was as follows: ¹H NMR (400 MHz, CD3OD) □ 6.82 (dd, 1H, J=5.31, 15.67 Hz), 5.85 (dd, 1H, J=1.77, 15.92 Hz), 4.61-4.52 (m, 1H), 4.33-4.25 (m, 2H), 4.15-4.05 (m, 2H), 3.25-3.20 (m, 2H), 2.51-2.38 (m, 1H), 2.26-2.17 (m, 1H), 2.00-1.93 (m, 1H), 1.91 (s, 3H), 1.78-1.41 (m, 8H), 1.23-1.14 (m, 3H), 0.94-0.82 (m, 12H).

### Example 14

### Preparation of Ethyl(2E,4S)-4-[(methylsulfonyl)amino]-5-[(3S)-2-oxopyrrolidin-3-yl]pent-2-enoate

A solution of ethyl(2E,4S)-4-amino-5-[(3S)-2-oxopyrrolidin-3-yl]pent-2-enoate in dichloromethane was prepared. One equivalent of methylsulfonyl chloride was then added followed by 1.1 equivalents triethyl amine and the solution was stirred at room temperature overnight. The reaction mixture was purified by flash chromatography on silica gel and eluted with 3% MeOH/CH₂Cl₂ and dried under high vacuum. The proton NMR was as follows: ¹H NMR, CDCl₃, 400 MHz: 6.82 (1H, dd, J=6.82, 15.66 Hz), 6.05-6.10 (2H, m), 4.19 (2H, q, J=7.07 Hz), 4.17 (1H, bs), 3.33-3.42 (2H), m), 2.92 (3H, s), 2.62 -2.70 (1H, m), 2.41 -2.49 (1H, m), 1.95 -2.04 (1H, m), 1.76-1.86 (2H, m), 1.59-1.65 (1H, m), 1.28 (3H, t, J=7.20 Hz).

### Example 15

### Preparation of Ethyl(2E,4S)-4-[(2-naphthylsulfonyl)amino]-5-[(3S)-2-oxopyrrolidin-3-yl]pent-2-enoate

A solution of ethyl(2E,4S)-4-amino-5-[(3S)-2-oxopyrrolidin-3-yl]pent-2-enoate in dichloromethane was prepared. One equivalent of naphthylene-2-sulfonyl chloride was then added followed by 1.1 equivalents triethyl amine and the solution was stirred at room temperature overnight. The reaction mixture was purified by flash chromatography on silica gel and eluted with 3% MeOH/CH₂Cl₂ and dried under high vacuum. The proton NMR was as follows: ¹H NMR, CDCl₃, 400 MHz: 8.39 (1H, s), 7.82 -7.94 (4H, m), 7.56 -7.65 (2H, m), 6.58 (1H, dd, J=6.57, 15.66 Hz), 6.01 (1H, bs), 5.84 (1H, dd, J=1.26, 15.67 Hz), 3.95 (3H, q, J=7.08 Hz), 3.13-3.29 (2H, m), 2.14-2.24 (2H, m), 1.89-1.97 (1H, m), 1.65-1.75 (1H, m), 1.48-1.55 (1H, m), 1.10 (3H, t, J=7.20 Hz).

While the invention has been described in terms of various preferred embodiments and specific examples, the invention should be understood as not being limited by the foregoing detailed description, but as being defined by the appended claims and their equivalents.

What is claimed is:

1. Compounds of the general formula 1 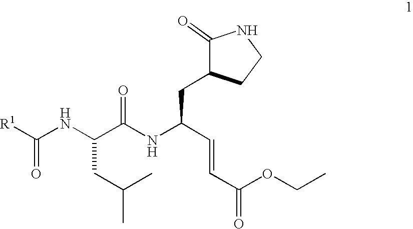
wherein:
R¹ is selected from C₁ to C₄ alkyl and 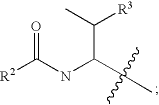
R² is selected from C₁ to C₄ alkyl and —OR⁵;
R⁵ is selected from C₁ to C₆ alkyl and
R³ is selected from halogen, —OH, —OR⁴ and C₁ to C₄ alkyl and to pharmaceutically acceptable salts and solvates thereof,

2. Compounds of the general formula 2 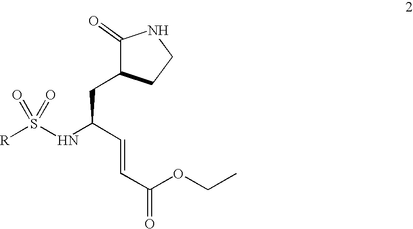
wherein R is C₁ to C₁₀ alkyl, aryl, heteroaryl, C₅ to C₁₀ cycloalkyl and C₄ to C₉ heterocycloalkyl and to pharmaceutically acceptable salts and solvates thereof.

3. A compound according to claim 1 selected from the group consisting of: 4-(2-Acetylamino-4-methyl-pentanoylamino)-5-(2-oxo-pyrrolidin-3-yl)-pent-2-enoic acid ethyl ester; 4-[2-(2-Acetylamino-3-hydroxy-butyrylamino)-4-methyl-pentanoylamino]-5-(2-oxo-pyrrolidin-3-yl)-pent-2-enoic acid ethyl ester, 4-[2-(2-tert-Butoxycarbonylamino-3-methyl-butyrylamino)-4-methyl-pentanoylamino]-5-(2-oxo-pyrrolidin-3-yl)-pent-2-enoic acid ethyl ester, 4-[2-(2-Acetylamino-3-methyl-butyrylamino)-4-methyl-pentanoylamino]-5-(2-oxo-pyrrolidin-3-yl)-pent-2-enoic acid ethyl ester, 4-[2-(2-tert-Butoxycarbonylamino-3-hydroxy-butyrylamino)-4-methyl-pentanoylamino]-5-(2-oxo-pyrrolidin-3-yl)-pent-2-enoic acid ethyl ester, 4-[2-Acetylamino-4-methyl-pentanoylamino)-4-methyl-pentanoylamino]-5-(2-oxo-pyrrolidin-3-yl)-pent-2-enoic acid ethyl ester.

4. The compound according to claim 3 wherein the compound is 4-(2-Acetylamino-4-methyl-pentanoylamino)-5-(2-oxo-pyrrolidin-3-yl)-pent-2-enoic acid ethyl ester.

5. The compound according to claim 3 wherein the compound is 4-[2-(2-Acetylamino-3-hydroxy-butyrylamino)-4-methyl-pentanoylamino]-5-(2-oxo-pyrrolidin-3-yl)-pent-2-enoic acid ethyl ester.

6. The compound according to claim 3 wherein the compound is 4-[2-(2-tert-Butoxycarboxylamino-3-methyl-butyrylamino)-4-methyl-pentanoylamino]-5-(2-oxo-pyrrolidin-3-yl)-pent-2-enoic acid ethyl ester.

7. The compound according to claim 3 wherein the compound is 4-[2-(2-Acetylamino-3-methyl-butyrylamino)-4-methyl-pentanoylamino]-5-(2-oxo-pyrrolidin-3-yl)-pent-2-enoic acid ethyl ester.

8. The compound according to claim 3 wherein the compound is 4-[2-(2-tert-Butoxycarbonylamino-3-hydroxy-butyrylamino)-4-methyl-pentanoylamino]-5-(2-oxo-pyrrolidin-3-yl)-pent-2-enoic acid ethyl ester.

9. The compound according to claim 3 wherein the compound is 4-[2-Acetylamino-4-methyl-pentanoylamino)-4-methyl-pentanoylamino]-5-(2-oxo-pyrrolidin-3-yl)-pent-2-enoic acid ethyl ester.

10. A compound according to claim 2 selected from the group consisting of

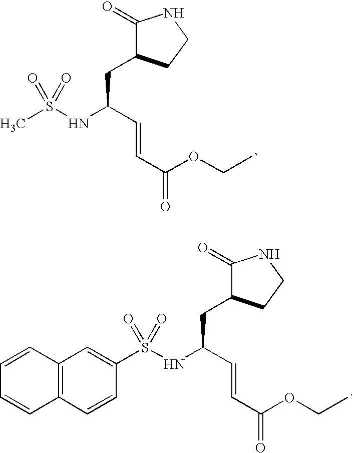

11. The compound according to claim 10 wherein the compound is

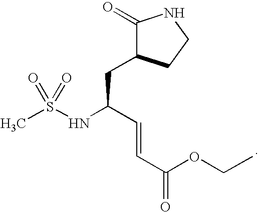

12. The compound according to claim 10 wherein the compound is [chemical structure]
